(19) United States
(12) Patent Application Publication
(10) Pub. No.: US 2006/0040935 A1
(43) Pub. Date: Feb. 23, 2006

(54) Method of treating impotence due to spinal cord injury

(75) Inventors: Murray C. Maytom, New York, NY (US); Ian H. Osterloh, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/257,797

(22) Filed: Oct. 24, 2005

Related U.S. Application Data
(63) Continuation of application No. 09/248,438, filed on Feb. 11, 1999.
(60) Provisional application No. 60/075,580, filed on Feb. 23, 1998.

(30) Foreign Application Priority Data
Feb. 23, 1998 (GB) 99301085.9

(51) Int. Cl. A61K 31/5377; A61K 31/519

(52) U.S. Cl. 514/234.5; 514/262.1; 514/252.16

(57) ABSTRACT

A class of cGMP PDE inhibitors, including sidenafil and pharmaceutically acceptable salts thereof, which can be used to treat sexual dysfunction in male and female animals, especially humans, with a spinal cord injury. The invention can be used to treat sexual dysfunction in male animals that exhibit essentially no residual penile function.

### FIELD OF THE INVENTION

This invention relates to a method of treating sexual dysfunction due to spinal cord injury (SCI) comprising administering an effective amount of a compound of formula I as defined below, including pharmaceutically acceptable salts thereof.

### BACKGROUND OF THE INVENTION

Impotence is the inability to obtain and/or sustain an erection sufficient for penetration of the vagina and/or intercourse. Thus, impotence is also referred to as “erectile insufficiency” or “erectile dysfunction”. It has been estimated that 10-12 million American men between the ages of 18 and 75 suffer from chronic impotence, with the great majority being over age 55.

The penis normally becomes erect when certain tissues, in particular the corpora cavernosa in the central portion of the penis, become engorged with blood, thereby causing them to become rigid, causing an erection. Impotence can result from psychologic disturbances (psychogenic), from physiologic abnormalities (organic) or from a combination of both. Thus, in some males erectile dysfunction may be due to anxiety or depression, with no apparent somatic or organic impairment. In other cases, erectile dysfunction is associated with atherosclerosis of the arteries supplying blood to the penis. In still other cases, the dysfunction may be due to venous leakage or abnormal drainage in which there is leakage from veins in the penis such that sufficient pressure for an erection can be neither obtained nor maintained. In still other cases, the dysfunction is associated with a neuropathy or due to nerve damage arising from, for example, surgery or a pelvic injury. Typically, multiple factors are responsible for impotence.

### SUMMARY OF THE INVENTION

This invention provides a method of treating sexual dysfunction in an animal with an injured spinal cord, comprising administering to an animal, particularly a human, in need of such treatment an effective amount of a compound of formula (I): 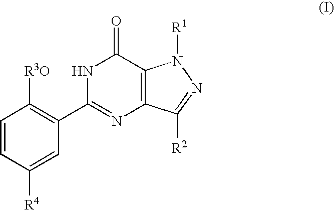
wherein:
R¹ is H; C₁-C₃ alkyl; C₁-C₃ perfluoroalkyl; or C₃-C₅ cycloalkyl;
R² is H; C₁-C₆ alkyl optionally substituted with C₃-C₆ cycloalkyl; C₁-C₃ perfluoroalkyl; or C₃-C₆ cycloalkyl;
R³ is C₁-C₆ alkyl optionally substituted with C₃-C₆ cycloalkyl; C₁-C₆ perfluoroalkyl; C₃-C₅ cycloalkyl; C₃-C₆ alkenyl; or C₃-C₆ alkynyl;
R⁴ is C₁-C₄ alkyl optionally substituted with OH, NR⁵R⁶, CN, CONR⁵R⁶ or CO₂R⁷; C₂-C₄ alkenyl optionally substituted with CN, CONR⁵R⁶ or CO₂R⁷; C₂-C₄ alkanoyl optionally substituted with NR⁵R⁶; (hydroxy)C₂-C₄ alkyl optionally substituted with NR⁵R⁶; (C₂-C₃ alkoxy)C₁-C₂ alkyl optionally substituted with OH or NR⁵R⁶; CONR⁵R⁶; CO₂R⁷; halo; NR⁵R⁶; NHSO₂NR⁵R⁶; NHSO₂R⁸; SO₂NR⁹R¹⁰; or phenyl, pyridyl, pyrimidinyl, imidazolyl, oxazolyl, thiazolyl, thienyl or triazolyl any of which is optionally substituted with methyl;
R⁵ and R⁶ are each independently H or C₁-C₄ alkyl, or together with the nitrogen atom to which they are attached form a pyrrolidinyl, piperidino, morpholino, 4-N(R¹¹)-piperazinyl or imidazolyl group wherein said group is optionally substituted with methyl or OH;
R⁷ is H or C₁-C₄ alkyl;
R⁸ is C₁-C₃ alkyl optionally substituted with NR⁵R⁶;
R⁹ and R¹⁰ together with the nitrogen atom to which they are attached form a pyrrolidinyl, piperidino, morpholino or 4-N(R¹²)-piperazinyl group wherein said group is optionally substituted with C₁-C₄ alkyl, C₁-C₃ alkoxy, NR¹³R¹⁴ or CONR¹³R¹⁴;
R¹¹ is H; C₁-C₃ alkyl optionally substituted with phenyl; (hydroxy)C₂-C₃ alkyl; or C₁-C₄ alkanoyl;
R¹² is H; C₁-C₆ alkyl; (C₁-C₃ alkoxy)C₂-C₆ alkyl; (hydroxy)C₂-C₆ alkyl; (R¹³R¹⁴N)C₂-C₆ alkyl; (R¹³R¹⁴NOC)C₁-C₆ alkyl; CONR¹³R¹⁴; CSNR¹³R¹⁴; or C(NH)NR¹³R¹⁴; and
R¹³ and R¹⁴ are each independently H; C₁-C₄ alkyl; (C₁-C₃ alkoxy)C₂-C₄ alkyl; or (hydroxy)C₂-C₄ alkyl;
or a pharmaceutically acceptable salt thereof;
or a pharmaceutical composition containing either entity.

The above compounds are disclosed, inter alia, in U.S. Pat. Nos. 5,250,534, 5,272,147 and 5,426,107, all herein incorporated by reference, and in WO 94/28902.

Types of sexual dysfunction due to spinal cord injury which are treatable by means of this invention include male erectile dysfunction and female sexual dysfunction such as orgasmic dysfunction and arousal disorders.

“Sexual dysfunction in an animal with an injured spinal cord” means sexual dysfunction in an animal due to the trauma and/or nerve damage which accompanies a physical spinal cord injury or nerve damage resulting from organic disease. In this type of injury the cortical components of sexual arousal (for example visual sexual stimulation) are disassociated from the localized reflexogenic component of the arousal process. There are, of course, varying degrees of spinal cord injury. The average male patient suffers nerve damage sufficient to prevent the patient from being able to obtain and/or sustain an erection sufficient for intercourse, yet the patent still exhibits a reflexogenic erectile response. It is considered unique to administer an oral drug that only in the presence of tactile genital stimulation (as occurs in sexual foreplay) has the ability to prolong and enhance the normal reflexogenic response in this SCI patient population. The use of a compound according to the present invention can restore erectile function to the point that an SCI patient can sustain an erection sufficient for intercourse.

A subset of spinal cord injured patients includes male patients who have essentially no residual erectile function following the injury. Such a patient can be defined as one who exhibits no apparent erectile response, indicating no reflexogenic erectile response to local stimulation, usually penile vibratory stimulation (PVS), and no erections induced by other means (e.g., visual stimulation). It has been determined that use of a compound in accordance with this invention can restore erectile function sufficient for intercourse in a substantial proportion of this SCI patient population. It is truly surprising that erectile function can be restored in a patient who has sustained a SCI to the extent that, in the absence of treatment with a compound of formula (I), local stimulation produces no apparent erectile response.

### DETAILED DESCRIPTION

Reference to a compound of formula I, both in this disclosure and the appendant claims, shall at all times be understood to include all active forms of such compounds, including the free form thereof (e.g., the free add or base form) and also all pharmaceutically acceptable salts, prodrugs, polymorphs, hydrates, solvates, stereoisomers (e.g. diastereomers and enantiomers), and so forth. Active metabolites of such compounds are also included.

Preferred compounds of formula (I) include those which can be taken as required, as compared with needing to be taken chronically. Such preferred compounds include those which improve the sexual response such that the patient responds to sexual (e.g., visual and/or tactile) stimulation, as opposed to compounds which act by causing an erection in the absence of sexual stimulation.

Additional preferred compounds include those which are “fast acting”, meaning that the time taken from administration to the point at which the sexual response is improved is less than about two hours, preferably less than about one hour, more preferably on the order of a half hour or less, and even more preferably within 10 or 15 minutes.

Preferred compounds (which are cGMP PDEᵥ inhibitors) include sildenafil, 5-[2-ethoxy-5-(4-methyl-1-piperazinylsulphonyl)-phenyl]-1-methyl-3-n-propyl-1,6-dihydro-7H-pyrazolo[4,3-d]pyrimidin-7-one, which has the structure: 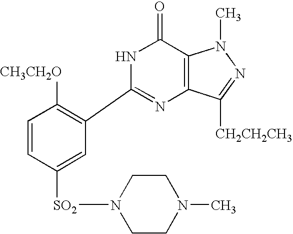
and pharmaceutically acceptable salts thereof, and the compound having the structure: 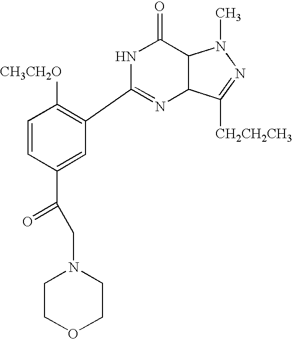
and pharmaceutically acceptable salts thereof. The first compound, sildenafil, is disclosed in U.S. Pat. No. 5,250,534, herein incorporated by reference. The second compound is disclosed, for example, in U.S. Pat. Nos. 5,272,147 and 5,426,107, both incorporated herein by reference.

A preferred pharmaceutically acceptable salt of sildenafil for use in this invention is the citrate salt, disclosed in co-pending U.S. provisional Application No. 60/027,690 filed Oct. 8, 1996 and incorporated herein by reference.

Other preferred compounds of formula (I) include those compounds selected from:
5-(5-morpholinoacetyl-2-n-propoxyphenyl)-methyl-3-n-propyl-1,6-dihydro-7H-pyrazolo[4,3-d]pyrimidin-7-one;
5-[2-allyloxy-5-(4-methyl-1-piperazinylsulphonyl)-phenyl]-1-methyl-3-n-propyl-1,6-dihydro-7H-pyrazolo[4,3-d]pyrimidin-7-one;
5-{2-ethoxy-5-[4-(2-propyl)-1-piperazinyl-sulphonyl]phenyl}-methyl-3-n-propyl-1,6-dihydro-7H-pyrazolo[4,3-d]pyrimidin-7-one;
5-{(2-ethoxy-5-[4-(2-hydroxyethyl)-1-piperazinyl-sulphonyl]phenyl}-methyl-3-n-propyl-1,6-dihydro-7H-pyrazolo[4,3-d]pyrimidin-7-one;
5-{5-[4-(2-hydroxyethyl)-1-piperazinylsulphonyl]-2-n-propoxyphenyl}-1-methyl-3-n-propyl-1,6-dihydro-7H-pyrazolo[4,3-d]pyrimidin-7-one;
5-[2-ethoxy-5-(4-methyl-1-piperazinylcarbonyl)-phenyl]-1-methyl-3-n-propyl-1,6-dihydro-7H-pyrazolo[4,3-d]pyrimidin-7-one; and
5-[2-ethoxy-5-(1-methyl-2-imidazoyl)phenyl]-1-methyl-3-n-propyl-1,6-dihydro-7H-pyrazolo[4,3-d]pyrimidin-7-one.

The above compounds are disclosed in the aforementioned U.S. Pat. Nos. 5,250,534, 5,272,147 and 5,426,107.

A compound of formula I will generally be administered via any of the known routes of administration such as oral, parenteral via local injection intracavemosally or intraurethrally, or transdermal as by applying the active component in a gel or other such formulation topically to the penis. Oral administration is preferred. The compound can be formulated as known in the art, usually together with a pharmaceutically acceptable carrier or diluent, for example as a tablet, capsule, lozenge, troche, elixir, solution, or suspension for oral administration, in a suitable injectable vehicle for parenteral administration, or as a lotion, ointment or cream for topical application.

The exact dose administered will, of course, differ depending on the specific compound of formula I prescribed, on the subject being treated, on the severity of the organic dysfunction, on the manner of administration and on the judgment of the prescribing physician. Thus, because of patient-to-patient variability, the dosages given below are a guideline and the physician may adjust doses of the compounds to achieve the treatment that the physician considers appropriate for the patient. In considering the degree of treatment desired, the physician must balance a variety of factors such as the age and sex of the patient and the presence of other diseases or conditions (e.g., cardiovascular disease). In general, the compound of formula I will be administered in a range of from 10 to 200 mg, preferably 25 to 100 mg, taken as required not more than once daily. Usually, the compound will be taken on demand, anywhere from a few minutes up to several hours prior to intercourse. As previously noted, a compound of formula I can be administered in any conventional oral, parenteral, rectal or transdermal dosage form, usually also together with a pharmaceutically acceptable carrier or diluent.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, a compound of formula I can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easter, Pa., 15th Edition (1975).

As an example of the invention, a study was conducted which had a double-blind, randomised, placebo-controlled, single dose, two-way crossover design. After a screening period in which only patients with at least a grade 2 (i.e., hard, but not hard enough for vaginal penetration) reflexogenic erectile response to a vibrator were included, fasted patients were randomly allocated to receive a single oral dose of 50 mg of sildenafil or placebo, administered an double-blind fashion in a private room; a washout period of 3 days was used between the crossover periods.

Twenty-seven male patients (mean age 32.9 years, range 21-49 years) with erectile dysfunction solely attributable to a spinal cord injury (cord level range T6-L4/5) were studied. One patient did not complete the study.

Reflexogenic erections were stimulated by applying a vibrator to the shaft and glans of the penis at set times: T=0 (pre-dose), and at T=0.5 hour, T=1 hour, and T=1.5 hours. Efficacy was evaluated by RigiScan® penile plethysmography recordings.

Twenty six patients were evaluable. No patients discontinued treatment due to adverse events. The results of the RigiScan® assessments (Stage I) and the primary efficacy analysis question answered at the end of the 28-day treatment period (Stage II) are shown in tables A and B immediately below:

STAGE I: single-dose, two-way crossover study
RigiScan ® recordings (n = 26)

| | No. patients (%) with |
|---|---|
| | penile BASE rigidity >60% |
| SILDENAFIL | 17/26 (65%)* |
| PLACEBO | 2/26 (8%) |

*significantly different from placebo, p < 0.01

STAGE II: 28-day, parallel-group study
†Has the treatment you have been taking over
the last 4 weeks improved your erections?

| | YES | NO |
|---|---|---|
| SILDENAFIL (n = 12) | 9/12 (75%)** | 3/12 (25%) |
| PLACEBO (n = 14) | 1/14 (7.1%) | 13/14 (92.9%) |

**significantly different from placebo, p < 0.01

1. (canceled)

2. A method of treating sexual dysfunction due to trauma and/or nerve damage which accompanies a spinal cord injury in an animal, comprising orally administering to an animal in need of such treatment an effective amount of sildenafil, having the structure: 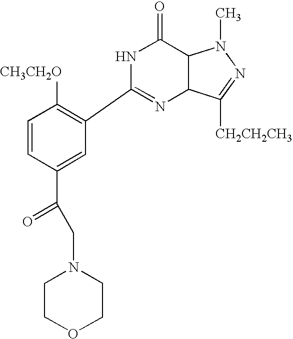
and pharmaceutically acceptable salts thereof.

3. (canceled)

4. (canceled)

5. A method of treating sexual dysfunction due to trauma and/or nerve damage which accompanies a spinal cord injury in an animal, comprising orally administering to an animal in need of such treatment an effective amount of sildenafil, or a pharmaceutically acceptable salt thereof, or a pharmaceutical composition containing either entity.

6. A method as defined in claim 5, wherein said pharmaceutically acceptable salt is the citrate.

7. (canceled)

8. A method as defined in claim 5, wherein said animal is male and exhibits essentially no residual erectile function.

9. (canceled)

10. A method as defined in claim 5, wherein said animal is human.
